(19) United States
(12) Patent Application Publication
(10) Pub. No.: US 2023/0337614 A1
(43) Pub. Date: Oct. 26, 2023

(54) METHOD FOR BREEDING FRESH-EATING WAXY CORN VARIETY WITH SWEET TASTE

(71) Applicant: Beijing Academy of Agriculture and Forestry Sciences, Beijing (CN)

(72) Inventors: Jiuran Zhao, Beijing (CN); Boshan Lu, Beijing (CN); Yaxing Shi, Beijing (CN); Li Xu, Beijing (CN)

(21) Appl. No.: 17/969,828

(22) Filed: Oct. 20, 2022

(30) Foreign Application Priority Data
Apr. 21, 2022 (CN) 202210419018X

(51) Int. Cl. A01H 6/46; A01H 5/10

(52) U.S. Cl. CPC A01H 6/4684; A01H 5/10

(57) ABSTRACT

A method for breeding a fresh-eating waxy corn variety with sweet taste is provided, relates to the technical field of fresh-eating waxy corns. According to the method, a waxy corn cultivar is hybridized with a supersweet waxy homozygous double-recessive backbone self-line to obtain a waxy corn self-line with sweet taste A, and the waxy corn self-line with sweet taste A and a waxy corn backbone self-line are performed with hybridization group to obtain the fresh-eating waxy corn variety with sweet taste. The breeding method can create an excellent waxy self-line with sweet taste having high seed germination rate, good plant agronomic traits, strong resistance and high sugar content in kernels. The sugar content and taste quality of the bred waxy corn with sweet taste are significantly improved and the harvest period is much longer, which is suitable for fresh-eating and quick-freezing processing.

### TECHNICAL FIELD

The disclosure relates to the technical field of fresh-eating waxy corns, and more particularly to a method for breeding a fresh-eating waxy corn variety with sweet taste.

### BACKGROUND

Waxy corn (Zea mays L. var. ceraina Kulesh) is a special corn type formed by recessive mutation (i.e., from Waxy to waxy) of an endosperm gene in kernels of a normal corn (also referred to as normal maize). Compared with the normal corn, almost 100% of starch contained in waxy corn kernels is amylopectin, nutrient substances are rich, planting benefit is high, and the waxy corn has become an important crop for adjusting a structure of cropping in China and driving increase of farmers' income.

The waxy corn tastes sticky, soft and fragrant, but sugar content in kernels is low, the sugar content in the kernels (also expressed to as Brix value, in percentage %) in a milk ripening stage is generally about 8%, and there is no sweet taste. In order to meet diversified demands of the market, a sweet-waxy corn has been bred in China, the sweet-waxy corn has both sweet and waxy kernels on one ear, and the two kinds of kernels exist independently. Compared with the pure waxy corn, one the one hand, an average sweetness of ears of the sweet-waxy corn is improved while is not suitable for quick-freezing processing. On the other hand, breeding of the common sweet-waxy corn in production at present requires to create sweet and waxy double recessive self-line (sh2sh2wxwx), with a complex process and a long breeding period. In addition, because of the low starch content of kernels, there are some problems such as low germination rate, weak emergence and poor resistance.

### SUMMARY

An objective of the disclosure is to provide a pure waxy corn with a high sugar content.

In a first aspect, the disclosure provides a method for breeding a fresh-eating waxy corn variety with sweet taste, including:
hybridizing a waxy corn cultivar with a super sweet waxy homozygous double-recessive backbone self-line, identifying and selecting ears with sugar contents of kernels being greater than 12%, and simultaneously performing selfing and grading identification for continuous generations, and obtaining a waxy corn self-line with sweet taste A; and
performing hybridization group on the waxy corn self-line with sweet taste A with a waxy corn backbone self-line to obtain ears of an offspring, and identifying and selecting a hybridization combination, from the ears of the offspring, with a sugar content of kernels of ears being greater than 12%, to obtain the fresh-eating waxy corn variety with sweet taste.

In the related art, on the one hand, normal waxy corn is not sweet in taste and cannot meet diversified demands of the market; and on the other hand, a way to achieve both sweet and waxy taste is that the sweet corn and the waxy corn are hybridized to form a sweet-waxy type corn, and ears of the type corn contain both waxy kernels and sweet kernels. Due to a fact that the sweet kernels are contained, a harvest period of the sweet-waxy corn is short, and in the storage and transportation and quick-freezing processing process, it is easy to appear problems of color difference, uneven stick surface, easy breakage of sweet kernels and the like, resulting in the reduction of appearance regularity. Therefore, the sweet-waxy corn is mainly used for fresh-eating, but is not suitable for quick-freezing processing.

Compared with the normal waxy corn, the waxy corn with sweet taste provided by the disclosure has higher sugar content in kernels and obvious sweet taste. Compared with the sweet-waxy corn, ears of the waxy corn with sweet taste provided by the disclosure are full of waxy kernels, with a long harvest period and neat appearance, which is suitable for quick-freezing processing and transportation.

At present, main types of fresh-eating corn are sweet corn, waxy corn and sweet-waxy corn, and there is no waxy corn with sweet taste. The waxy corn with sweet taste mentioned in the disclosure is innovatively created by the applicant and is a new type of fresh-eating waxy corn. In the breeding process of the related art, destructive sampling is often selected for identification of indexes, and such sampling and identification manners often cause the contradiction that kernels cannot be taken for detection in order to obtain selfed ears, or selfed ears cannot be obtained after kernels are taken.

In the breeding method provided by the disclosure, the identification of the waxy corn self-line with sweet taste A is as follows: taking down a middle-upper part of an ear, and continuously growing a middle-lower part of the ear to be a mature ear; threshing the taken middle-upper part of the ear to obtain waxy corn kernels, detecting a sugar content of the waxy corn kernels by using a Brix meter, and retaining the mature ear with a sugar content being greater than 12%.

The sampling and identification manners in the method of the disclosure not only complete the detection and screening target, but also obtain the mature selfing kernels; and breaks through the contradiction, in the past, that the kernels cannot be taken in order to obtain selfed ears, and the selfed ears cannot be obtained after the kernels are taken.

In the breeding method provided by the disclosure, a period for identifying the sugar content of the kernels of the ear is a milk ripening stage of the ear; and in an embodiment, the period for identifying the sugar content of the kernels of the ear may be 20-26 days after pollination.

In the method provided by the disclosure, the method further includes: performing hybridization group on the waxy corn self-line with sweet taste A with the waxy corn backbone self-line to obtain ears of an offspring, identifying and selecting a combination, from the ears of the offspring, with a sugar content of kernels of ears being greater than 12% at a milk ripening stage or 20-26 days after pollination and with a water content being greater than 50% after the pollination for 31 days as the fresh-eating waxy corn variety with sweet taste.

Varieties that can maintain greater than 50% water content after the pollination for 31 days can greatly increase the fresh-eating harvest period of waxy corn with high sugar content.

In the breeding method provided by the disclosure, a genotype of the supersweet waxy homozygous double-recessive backbone self-line is sh2sh2wxwx; and in an embodiment, the supersweet waxy homozygous double-recessive backbone self-line may be D6644.

In the breeding method provided by the disclosure, the fresh-eating waxy corn variety with sweet taste enters the harvest period after the pollination for 20 days.

In the breeding method provided by the disclosure, the waxy corn cultivar is Zhongnuo No. 2.

In a specific embodiment of the disclosure, the disclosure provides a method for breeding a fresh-eating waxy corn variety with sweet taste, including following steps.

Step (1), selfing by taking a cultivar of Zhongnuo No. 2 as a basic material to obtain a 51 population, and selecting a superior plant from the 51 population for selfing one generation to obtain a S2 population.

Step (2), selecting a superior plant from the S2 population, then hybridizing the selected superior plant from the S2 population with the supersweet waxy homozygous double-recessive backbone self-line, to obtain hybrid F1 seeds, planting the hybrid F1 seeds to obtain F1 plants, identifying and selecting superior individual plants with good growth, strong resistance and good green retention from the F1 plants, and performing selfing on the superior individual plants, so as to obtain selfed ears.

In the disclosure, the sweet and waxy double-recessive self-line is selected for hybridization instead of sweet or waxy corn self-lines. On the one hand, the sugar content of the supersweet waxy double-recessive self-line is higher than that of the sweet corn self-line and the waxy corn self-line, so that the probability of obtaining kernels with high sugar content in the offspring of hybridization and selfing is increased. On the other hand, the supersweet waxy double-recessive self-line contains wx homozygous genes, and the hybrid offspring still contains waxy kernels, which will not produce segregation, thus speeding up the breeding process.

Step (3), selecting waxy kernels of the selfed ears obtained in the step (2) and planting the waxy kernels to selfed plants, identifying and selecting superior individual plants with good growth, strong resistance and good green retention from the selfed plants for bagging and selfing, and detecting sugar contents of the waxy corn kernels at a milk ripening period of ears to retain the mature ears with the sugar contents being greater than 12%.

Step (4), repeating the step (3), then identifying and selecting pure waxy excellent ears with the sugar contents of the waxy kernels being greater than 13% and ear kernels having no separation, and continuously to self to obtain the waxy corn self-line with sweet taste A.

Sugar content in corn is a quantitative trait, which is controlled by micro genes. High sugar content is detected in the waxy corn kernels, which means that other minor genes are introduced into waxy kernels to increase sugar content. Therefore, the sugar content of waxy corn can be inherited in the process of hybridization and selfing and from a phenotype.

Step (5), hybridizing the waxy corn self-line with sweet taste A as a parent and the waxy corn backbone self-line as another parent, identifying and selecting a hybridization combination with a sugar content of kernels of ears at a milk ripening stage being greater than 12% and with the water content of the kernels of ears being greater than 50% after the pollination for 31 days, so as to obtain the fresh-eating waxy corn variety with sweet taste.

When the hybridization group is performed by using the waxy corn self-line with sweet taste A as the parent, the waxy corn backbone line must be selected as another parent, which can increase desirable gene frequency of offspring.

In the step (3), the milk ripening period is 20-26 days after the pollination; and in the step (4), the step (3) may be repeated twice or more.

In this method, ears with sugar content of greater than 12% in kernels are selected for more than two consecutive generations, and corresponding selfed ears are obtained at the same time; which can increase the selection frequency of self-lines with high sugar content, and can accumulate sugar content.

According to the understanding of those skilled in the art, the disclosure also requests to protect the waxy corn with sweet taste obtained by the above breeding method can be used in any of the following applications:

(1) improving the sweetness of the waxy corn; and (2) increasing the stability of the waxy corn with high sweetness in processing and transportation.

The breeding method provided by the disclosure can improve the sweetness of waxy corn, and can solve the problems that waxy corn is not sweet in taste and the harvest period is short in the related art.

The breeding method provided by the disclosure can increase the stability of the waxy corn with high sweetness in the processing and transportation, and solve the problems that in the related art, although the sweet-waxy corn is sweet in taste, the harvest period is short, the transportation is difficult, and the processing is not suitable.

In summary, the waxy corn with sweet taste provided by the disclosure can meet the requirements of people who like both sweet and waxy corn, and has wider applications/uses.

Beneficial effects of the disclosure are as follows.

1, the method provided by the disclosure takes waxy kernels and plants as research objects, does not involve breeding sweet and waxy double-recessive self-line, and the breeding process is simpler, more efficient, time-saving and labor-saving.

2, the method provided by the disclosure does not require destructive sampling detection, the selfing kernels can be obtained at the present generation and the detection can be performed at the same generation, and materials which are not easy to obtain in the breeding process are saved.

3, the method provided by the disclosure can create an excellent waxy self-line with sweet taste, which has high kernel germination rate, good plant agronomic traits, strong resistance, and high sugar content in kernels. A new type of fresh-eating waxy corn variety with sweet taste is bred, and the sugar content in the kernels reaches 14%. Compared with the normal waxy corn, the taste quality is obviously improved, and the harvesting period is greatly prolonged compared with the sweet-waxy corn; and the yield of hybrid is high. It is suitable for applications such as fresh-eating and quick-freezing processing of ears, and can meet the diversified demands of production and market.

### DETAILED DESCRIPTION OF EMBODIMENTS

The following embodiments are used to describe/illustrate the disclosure, but are not intended to limit the scope of the disclosure.

### Embodiment 1, Breeding of Waxy Self-Line with Sweet Taste

A breeding process of a waxy excellent self-line with sweet taste is provided in this embodiment, including steps as follows.

Step (1), selfing by taking a cultivar of Zhongnuo No. 2, an excellent waxy corn variety in China, as a basic material to obtain a S1 population, and selecting a superior plant from the S1 population for selfing one generation to obtain a S2 population.

Step (2), selecting a superior plant from the S2 population, then hybridizing the selected superior plant from the S2 population with a supersweet waxy homozygous double-recessive backbone self-line D6644 independently bred by Maize Research Center, Beijing Academy of Agriculture and Forestry Sciences, to obtain hybrid seeds of first-generation.

Step (3), plant the hybrid seeds of first-generation to obtain hybrid plants, and identifying and selecting a superior individual plant with good growth, strong resistance and good green retention from the hybrid plants for bagging and selfing to thereby obtain selfed ears.

Step (4), identifying and selecting waxy kernels from the selfed ears for planting to obtain selfed plants, and identifying and selecting a superior individual plant with good growth, strong resistance and good green retention from the selfed plants for bagging and selfing. At a milk ripening period of ears (i.e., 20-26 days after pollination), a middle-upper part of the selfed ear is taken down, and a middle-lower part of the selfed ear is continued to grow to be a mature ear. A sugar content of waxy corn kernels threshed from the taken middle-upper part of the selfed ear is detected, and the corresponding mature ears are retained when the sugar content is greater than 12%.

Step (5), repeating the above step (4) for two generations, identifying and selecting pure waxy excellent ears whose waxy kernels contain greater than 13% sugar content and the whole ear kernels have no separation, and continuously to selfing for two generations to create an excellent waxy corn self-line with sweet taste A.

The excellent waxy self-line with sweet taste A obtained in this embodiment has waxy kernels, high germination rate, neat and consistent plants, excellent comprehensive agronomic traits, semi-compact plant type, dark green leaves, and vigorous plant growth. The plant height is 165 centimeters (cm), the ear height is 73 cm, the male and female flowers period is coordinated, and the fecundity (also referred to as rate of bearing kernel) of the plant is good. The ear is cylinder-shaped, with a length of 10.5 cm, a thickness of 4.3 cm, 14-16 rows per ear, and waxy kernels. The seed germination rate is 95%, the growth vigor of the seedlings is strong, and the lodging resistance is strong. The sugar content of kernels at the milk ripening stage is 14.6%.

In this embodiment, the identification and selecting of sugar content of the self-line is as follows. At the milk ripening stage (i.e., 20-26 days after pollination), the upper half of ear is taken down to detect the sugar content; and the taken ear is tasted and identified at the same time. Through the detection and taste identification of more than 500 individual ears, it is found that when the sugar content is below 11%, the kernels had no sweet taste; when the sugar content is above 12%, the kernels begin to have sweet taste. According to the degree of sweetness, it can be divided into 1-grade sweetness (i.e., sugar content is in a range of 12-13%) and 2-grade sweetness (i.e., sugar content is greater than 13%), and the taste sweetness is very obvious when it is 2-grade sweetness.

For the first identification, the sugar content of 12% and above is taken as the selection standard to increase the selection frequency; and for the second and subsequent identification, the sugar content of more than 13% is taken as the selection standard, and thus the best is selected from the best. Finally, the excellent waxy corn self-line with sweet taste is selected, with a sugar content of 14.6% in the kernels, and the comprehensive agronomic traits are excellent.

### Embodiment 2, First Breeding of Fresh-Eating Waxy Corn Hybrid with Sweet Taste

Step (1), the excellent waxy corn self-line with sweet taste A obtained in the embodiment 1 is taken as a male parent (also referred to as father plant or father parent) and the waxy corn backbone self-line in China is taken as a female parent (also referred to as female plant) to hybridize, so as to obtain hybrid seeds.

Step (2), after the hybrid seeds are planted, comprehensive trait identification is first conducted to identify combinations with strong plants, dark green leaves, good bract green degree and strong resistance. Then, the sugar content in kernels is detected at the milk ripening stage of ears (i.e., 20-26 days after pollination) to select combinations with sugar content above 12%. Subsequently, the water content of the ears is detected after pollination for 27-31 days to select combinations with the kernel water content still above 50% after pollination for 31 days.

Step (3), Jingke Tiannuo 789, an excellent variety, is bred through the above detection and selection, and in the vegetative growth stage, the plant grows vigorously, the plant and bract have good green retention and strong resistance. The plant height is 270 cm, the ear height is 99 cm, the ear type is long and cylindrical, the ear length is 24 cm, the ear thickness is 5.4 cm, and the number of rows per ear is in a range of 14-16. The sugar content of kernels at the milk ripening stage (i.e., 20-26 days after pollination) reaches 14.1%, and the yield of fresh ears is outstanding, reaching more than 1200 kilograms per mu (kg/mu, where one mu is about 666.667 square meters). As a control, Nongkeyu 368, a sweet and waxy hybrid, has a yield of about 900 kg per mu. On the 31st day after pollination, the kernel water content of Jingke Tiannuo 789 is 55%, and the harvest period is more than 10 days.

Specifically, in this embodiment, the hybrid selection criteria are as follows.

At the milk ripening stage (i.e., 20-26 days after pollination), three ears are randomly selected, and middle kernels are taken respectively to measure the sugar content of the kernels. The combination is selected when the kernel sugar content is greater than 12%; In addition to the sugar content, ear flavor is also an important trait for variety identification. Therefore, further, the ear flavor is taken as a second index for taste identification, and the combination is selected when the flavor score is above 8 (a total score is set to 10). Subsequently, from the 27th day after pollination, three ears are randomly taken every day, and middle kernels of the ears are taken to measure the kernel water content; on the 31th day after pollination, when the kernel water content is more than 50%, the combination is retained. In this situation, the combination has soft and tender taste, sweet taste and long harvest period.

### Embodiment 3, Second Breeding of Fresh-Eating Waxy Corn Hybrid with Sweet Taste

Step (1), the excellent waxy corn self-line with sweet taste A obtained in the embodiment 1 is taken as a female parent and the waxy corn backbone self-line in China is taken as a male parent to hybridize, so as to obtain hybrid seeds.

Step (2), after the hybrid seeds are planted, comprehensive trait identification is first conducted to identify combinations with strong plants, dark green leaves, good bract green degree and strong resistance. Then, the sugar content in kernels is detected at the milk ripening stage of ears (i.e., 20-26 days after pollination) to select combinations with sugar content above 12%. Subsequently, the water content of the ears is detected after pollination for 27-31 days to select combinations with the kernel water content still above 50% after pollination for 31 days.

Step (3), an excellent variety H is bred through the above detection and selection, the plant grows vigorously, the plant and bract have good green retention and strong resistance. The plant height is 235 cm, the ear height is 98 cm, the ear is long and cylindrical, the ear length is 22 cm, the ear thickness is 5.3 cm, and the number of rows per ear is in a range of 14-16. The sugar content of kernels at the milk ripening stage (i.e., 20-26 days after pollination) reaches 13.8%, and the yield of fresh ears is more than 1100 kg/mu, while the yield of the Nongkeyu 368 of sweet and waxy variety is about 900 kg/mu. On the 31st day after pollination, the kernel water content of the variety H is 54%, and the harvest period is more than 10 days.

Although the disclosure has been described in detail above with general description and specific embodiments, on the basis of the disclosure, it is apparent to those skilled in the art that various modifications and improvements may be made thereto. Therefore, these modifications or improvements made on the basis of without deviating from the spirit of the disclosure shall fall within the protection scope of the disclosure.

1. A fresh-eating waxy corn variety breeding method, comprising:
hybridizing a waxy corn cultivar with a supersweet waxy homozygous double-recessive backbone inbred line, identifying and selecting ears with sugar contents of the kernels are greater than 12%, simultaneously performing selfing and grading identification for continuous generations, and obtaining a waxy corn inbred line A with at least 12% sugar content;
wherein the grading identification of the waxy corn inbred line A with at least 12% sugar content comprises:
removing the middle-upper part of each of the ears, and continuously growing the middle-lower part of each of the ears to be a mature ear; and
threshing the middle-upper part of each of the ears to obtain waxy corn kernels of each of the ears, detecting a sugar content of the waxy corn kernels of each of the ears, and retaining the mature ear corresponding to the sugar content of the waxy corn kernels being greater than 12%; and
performing hybridization on the waxy corn inbred line A with at least 12% sugar content and a waxy corn backbone inbred line to obtain ears of an offspring, and identifying and selecting a hybridization combination, from the ears of the offspring, with a sugar content of kernels of ears being greater than 12% at the milk ripening stage or 20-26 days after pollination and with a water content of the kernels of ears being greater than 50% at 31 days after the pollination as a fresh-eating waxy corn variety;
wherein the genotype of the supersweet waxy homozygous double-recessive backbone inbred line is presented as shrunken-2(sh2)sh2waxy(wx)wx.

2. (canceled)

3. The method according to claim 1, wherein the waxy corn cultivar is Zhongnuo No. 2.

4-6. (canceled)

7. A fresh-eating waxy corn variety breeding method, comprising:
step (1), selfing by taking a waxy corn cultivar as a basic material to obtain a S1 population, and selecting a plant from the S1 population for selfing one generation to obtain a S2 population;
step (2), selecting a plant from the S2 population, then hybridizing the selected plant from the S2 population with a supersweet waxy homozygous double-recessive backbone inbred line to obtain hybrid F1 seeds, planting the hybrid F1 seeds to obtain F1 plants, identifying and selecting individual plants from the F1 plants, and performing selfing on the individual plants to obtain selfed ears; wherein the genotype of the supersweet waxy homozygous double-recessive backbone self-line inbred line is presented as sh2sh2wxwx;
step (3), selecting waxy kernels of the selfed ears obtained in the step (2) and planting the waxy kernels to obtain selfed plants, identifying and selecting individual plants from the selfed plants for bagging and selfing, and detecting sugar contents of waxy corn kernels at a milk ripening period of ears to retain mature ears with sugar contents being greater than 12%;
step (4), repeating the step (3), then identifying and selecting a plant containing pure waxy ears with sugar contents of waxy kernels being greater than 13%, and continuously to selfing the plant to obtain the waxy corn inbred line A; and
step (5), hybridizing the waxy corn inbred line A as a parent and a waxy corn backbone inbred line as another parent, identifying and selecting a hybridization combination with sugar contents of kernels of ears at the milk ripening stage being greater than 12% and with a water content of the kernels of ears 31 days after the pollination being greater than 50%, to obtain a fresh-eating waxy corn variety.

8. The method according to claim 7, wherein in the step (3), the milk ripening stage is 20-26 days after the pollination; and in the step (4), the step (3) is repeated twice or more.

9. The method according to claim 7, wherein the waxy corn cultivar is Zhongnuo No. 2.

10. A fresh-eating waxy corn variety breeding method, comprising:
step (1), selfing by taking a waxy corn cultivar as a basic material to obtain a S1 population, and selecting a plant from the S1 population for selfing one generation to obtain a S2 population;
step (2), selecting a plant from the S2 population, then hybridizing the selected plant from the S2 population with a supersweet waxy homozygous double-recessive backbone inbred line to obtain hybrid F1 seeds, planting the hybrid F1 seeds to obtain F1 plants, identifying and selecting individual plants from the F1 plants, and performing selfing on the individual plants to obtain selfed ears; wherein the genotype of the supersweet waxy homozygous double-recessive backbone self-line inbred line is presented as sh2sh2wxwx;
step (3), selecting waxy kernels of the selfed ears obtained in the step (2) and planting the waxy kernels to obtain selfed plants, identifying and selecting individual plants from the selfed plants for bagging and selfing, and detecting sugar contents of waxy corn kernels at a milk ripening period of ears to retain mature ears with the sugar contents being greater than 12%;
step (4), repeating the step (3), then identifying and selecting a plant containing pure waxy ears with sugar contents of waxy kernels being greater than 13%, and continuously to selfing the plant to obtain the waxy corn inbred line A; and
step (5), hybridizing the waxy corn inbred line A as a parent and a waxy corn backbone inbred line as another parent different from the supersweet waxy homozygous double-recessive backbone inbred line, identifying and selecting a hybridization combination with sugar contents of kernels of ears at the milk ripening stage being greater than 12% and with a water content of the kernels of ears 31 days after the pollination being greater than 50%, to obtain a fresh-eating waxy corn variety.
